(19) United States
(12) Patent Application Publication
(10) Pub. No.: US 2021/0135268 A1
(43) Pub. Date: May 6, 2021

(54) ELECTRODE FOR BIOELECTRONICS USING METAL-IMMOBILIZED PEPTIDE EXPRESSING ENZYME

(71) Applicants: GWANGJU INSTITUTE OF SCIENCE AND TECHNOLOGY, Gwangju (KR); KOREA UNIVERSITY RESEARCH AND BUSINESS FOUNDATION, Seoul (KR)

(72) Inventors: In Seop CHANG, Gwangju (KR); Yoo Seok LEE, Gwangju (KR); In Geol CHOI, Seoul (KR); Seung Woo BAEK, Gwangju (KR)

(21) Appl. No.: 17/053,202

(22) PCT Filed: May 9, 2019
(86) PCT No.: PCT/KR2019/005585

§ 371 Date: Nov. 5, 2020

(30) Foreign Application Priority Data
May 9, 2018 (KR) 10-2018-0053083

(51) Int. Cl. H01M 8/16; C12N 11/14; H01M 4/90; C07K 7/08

(52) U.S. Cl. CPC H01M 8/16; C07K 7/08; H01M 4/9008; C12N 11/14

(57) ABSTRACT

An electrode for an enzymatic biofuel cell may include: a substrate; an electrode positioned on the substrate; and an enzyme pattern containing an enzyme in which a metal-immobilized peptide is expressed positioned on the electrode. The metal-immobilized peptide expressed at the enzyme active site strongly binds to metal deposited electrode surface. In this manner, the enzyme active site facing to electrode was controlled to be in close proximity such that electron transfer efficiency of the electrode can improve.

### TECHNICAL FIELD

The present invention relates to a biocatalyst-based electrode for bioelectronics, and more specifically, an electrode for bioelectronics which uses metal-immobilized peptide-expressed enzymes.

### BACKGROUND ART

An enzyme is a catalyst that mediates a chemical reaction inside a living organism. An enzyme binds to a substrate to form an enzyme-substrate complex, thereby functioning as a catalyst that lowers activation energy of a reaction. A general catalyst requires a high temperature, strong acid, or strong alkali environment in order to function as a catalyst, whereas an enzyme can act at room temperature or even under a normal pH condition. In addition, the enzyme fulfills a function of a catalyst in a complicated environment, that is, the inside of a living organism, thus, having the substrate selectivity. Consequently, there is a reduction in necessity of purifying a reactant in advance.

Such characteristics of enzyme is led to development of technologies in which the enzyme is used as a commonly used catalyst, and a representative example of the technologies is an enzymatic biofuel cell. The enzymatic biofuel cell can be used as a power source of a human-implantable medical device, thus, attracting much attention; however, a problem arises in that the enzymatic biofuel cell generates a small quantity of electric power. In particular, a size of the enzymatic biofuel cell needs to be decreased to several centimeters or smaller in order to be used as a human-implantable power source. In this case, a quantity of electric power to be generated further decreases, and thus it is difficult to supply sufficient electric power to the human-implantable medical device.

In order to improve performance of the enzymatic biofuel cell, performance of an enzyme electrode which is a core member needs to be improved. The enzyme electrode is manufactured by fixing enzymes to an electrode that conducts electricity well, and a step of fixing enzymes is an important step. Fixing of the enzymes to an electrode material needs to be performed with consideration for the following conditions in order to improve the performance. First, enzymes can be fixed to an electrode material generally through physical adsorption or chemical binding, and then long-term attachment needs to be achieved. Secondly, an active site which reacts with a substrate in an enzyme fixing step to cause a redox reaction needs to easily come into contact with the substrate present in a solution such that there is no limitation in performance. Thirdly, an enzyme needs to be fixed while a final-electron supply active site that transfers, outside an enzyme, electrons generated through a substrate redox reaction in the enzyme fixing step is located at a distance of about tens of nanometers from a material surface of an electrode or the like having high conductivity.

When enzymes are fixed to an electrode surface of an electrode for an enzymatic biofuel cell or the like, gold nanoparticles or a redox mediator having high conductivity is synthesized on an enzyme surface or the electrode surface such that electrons can be transferred between the electrode and the enzymes, and binding of highly reactive functional groups such as an amino group, a carboxyl group, a thiol group, and an aromatic hydroxy group is used.

In an enzyme immobilization method by such chemical synthesis, efficiency of an electrode can decrease due to problems occurring in that reactivity of an enzyme can decrease due to occurrence of unexpected chemical binding in the enzyme, non-specific binding between an enzyme and a mediator or a mediator and an electrode can occur in a solution such that a redox active site of an enzyme cannot be protected, enzymes are not uniformly distributed over an electrode surface, and a minimum distance between an electrode and an electron transferring active site of an enzyme is not secured.

### CITATION LIST

### Patent Literature

### [Patent Literature 1]

Korean Unexamined Patent Publication No. 10-2012-0113085

### SUMMARY OF INVENTION

### Technical Problem

A technical object to be achieved by the present invention is to solve the problems described above and to provide an electrode for an enzymatic biofuel cell in which direct electron transfer can be performed by controlling a distance between a surface of an electrode and an electron transferring active site of an enzyme to be short.

Another technical object to be achieved by the present invention is to provide an electrode for an enzymatic biofuel cell which has improved efficiency by adjusting a phase of enzymes and fixing the enzymes to a surface of an electrode and being capable of arranging the enzymes in one layer over the surface with specificity to an electrode without conglomerate of enzymes.

Technical objects to be achieved by the present invention are not limited to the technical objects mentioned above, and the following description enables other unmentioned technical objects to be clearly understood by a person of ordinary skill in the art to which the present invention belongs.

### Solution to Problem

In order to achieve the technical object, an embodiment of the present invention provides an electrode for an enzymatic biofuel cell.

In this case, the electrode for an enzymatic biofuel cell may include: a substrate; an electrode positioned on the substrate; and an enzyme pattern containing an enzyme in which a metal-immobilized peptide is expressed positioned on the electrode.

In this case, the metal-immobilized peptide expressed in the enzyme may be immobilized at the electrode.

In the electrode for an enzymatic biofuel cell, a distance between an electron transferring active site of the enzyme and a surface of the electrode may be 2 nm or shorter.

In this case, the substrate may contain a silicon wafer, conductive polymers, carbon cloth, carbon paper, or graphene.

In this case, the electrode may contain silica, Cu, Zn, Fe, Ni, Co, Mn, Au, or Ag.

In this case, the enzyme may contain an α unit at which an active site is positioned and a γ unit coupled to the α unit, and the peptide may be expressed at either the α unit or the γ unit.

In this case, the peptide may specifically bind to the electrode and may have a spiral structure.

In this case, the peptide may contain 12 to 60 amino acids.

In this case, the peptide may contain one or more amino acid sequences of Sequence Number 1 to Sequence Number 10.

In this case, the enzyme may include a glucose dehydrogenase, a glucose oxidase, an alkaline phosphatase, or a carbon monoxide dehydrogenase.

In this case, the enzyme may further include a cofactor.

In order to achieve the technical object described above, another embodiment of the present invention provides an enzymatic biofuel cell including the electrode for an enzymatic biofuel cell.

### Advantageous Effects of Invention

According to embodiments of the present invention, it is possible to provide an electrode for an enzymatic biofuel cell in which direct electron transfer can be performed by controlling a distance between an active site of an enzyme and a surface of an electrode to be short.

According to the embodiments of the present invention, it is possible to provide an electrode for an enzymatic biofuel cell which has improved efficiency by being capable of arranging enzymes at regular intervals over the surface of the electrode without conglomerate or arranging the enzymes in a pattern.

Effects of the present invention are construed not to be limited to the above-mentioned effects but to include every effect that can be derived from configurations of the present invention described in the detailed description of the embodiments or claims of the present invention.

### BRIEF DESCRIPTION OF DRAWINGS

FIG. 1 is a schematic view of an enzyme electrode at which a metal-immobilized peptide-expressed enzyme is nanopatterned and fixed to a pattern electrode modified to a size of nanometer according to an embodiment of the present invention.

FIG. 2 is a sequence and an SDS-gel photograph of the metal-immobilized peptide-expressed enzyme according to the embodiment of the present invention.

FIG. 3 is a schematic view illustrating an electrode for an enzymatic biofuel cell according to the embodiment of the present invention.

FIG. 4 is a schematic view illustrating an electrode for an enzymatic biofuel cell in the related art.

FIG. 5 illustrates AFM measurement graphs of a manufacturing example and a comparative example according to the present invention.

FIG. 6 illustrates a cyclic voltammogram of the manufacturing example and the comparative example according to the present invention.

FIG. 7 illustrates a cyclic voltammogram based on a scan speed according to the present invention.

### DESCRIPTION OF EMBODIMENTS

Hereinafter, the present invention will be described with reference to the accompanying drawings. However, the present invention can be realized as various different embodiments, thus not being limited to embodiments described here. Besides, a part irrelevant to the description is omitted from the drawings in order to clearly describe the present invention, and similar reference signs are assigned to similar parts through the entire specification.

In the entire specification, a case where a certain part “is coupled to (accesses, is in contact with, or is connected to)” another part includes not only a case where the parts are “directly coupled” to each other, but also a case where the parts are “indirectly coupled” to each other with another member interposed therebetween. In addition, a case where a certain part “includes” a certain configurational element means that another configurational element is not excluded but can be further included, unless specifically described otherwise.

Terms used in this specification are only used to describe a specific embodiment and are not intentionally used to limit the present invention thereto. Singular expressions also include a meaning of its plural expressions unless obviously implied otherwise in context. In this specification, words such as “to include” or “to have” are to be construed to specify that a feature, a number, a step, an operation, a configurational element, a member, or a combination thereof described in the specification is present and not to exclude presence or a possibility of addition of one or more other features, numbers, steps, operations, configurational elements, members, or combinations thereof in advance.

Hereinafter, embodiments of the present invention will be described in detail with reference to the accompanying drawings.

Hereinafter, an electrode for an enzymatic biofuel cell according to an embodiment of the present invention will be described.

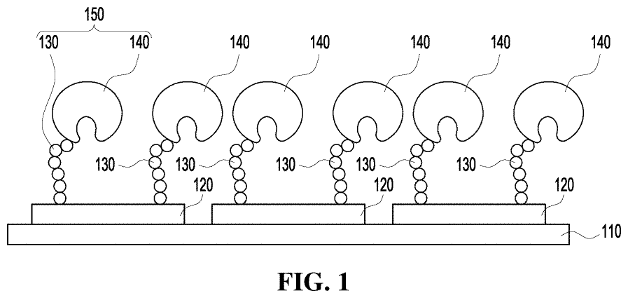

FIG. 1 is a schematic view illustrating the electrode for an enzymatic biofuel cell according to the embodiment of the present invention.

With reference to FIG. 1, an electrode 100 for an enzymatic biofuel cell can include a substrate 110, an electrode 120 positioned on the substrate 110, and an enzyme pattern 150 containing an enzyme 140 in which a metal-immobilized peptide 130 is expressed positioned on the electrode 120.

In this case, the metal-immobilized peptide (In other words, metal-binding peptide) 130 expressed at the enzyme 140 can be fixed at the electrode 120.

In the electrode for an enzymatic biofuel cell, a distance between an electron transferring active site of the enzyme and a surface of the electrode can be 2 nm or shorter.

Here, when the distance between the electron transferring active site and the surface of the electrode is longer than 2 nm, electron transfer resistance increases, and thus electrons may not be transferred.

The substrate 110 can fulfill functions of receiving and dispersing electrons and transferring the electrons to other parts of the biofuel cell.

In addition, the substrate fulfills a function of maintaining a shape of the electrode and thus needs to have machinability suitable for purposes of the biofuel cell. A final goal for the enzymatic biofuel cell is to have a large size like a general fuel cell; however, the study on the enzymatic biofuel cell mainly pursues, as a target, a power source for a bio-implantable medical device due to a limit of a fuel until today. Consequently, a technology for minimizing and nanomizing the biofuel cell needs to be provided, and the substrate needs to be made of a material that can be minimized depending on a size of the biofuel cell.

For example, the substrate 110 can contain a silicon wafer, conductive polymers, carbon cloth, carbon paper, or graphene.

In this case, the silicon wafer has high reliability and highly accurate machinability as mainly used in the semiconductor as a conductive material used for a long time or the like. On the other hand, the conductive polymers have an advantage of having high conductivity for a unit volume and being able to be machined into a desired shape. In addition, the carbon cloth, the carbon paper, the graphene, and the like have very high conductivity due to a carbon material. Here, a material forming the substrate is not limited thereto and can be variously selected depending on a size and type of target enzymatic biofuel cell.

For example, the electrode 120 can contain silica, Cu, Zn, Fe, Ni, Co, Mn, Au, or Ag.

For example, the electrode 120 can be a pattern-formed electrode. In this case, the pattern-formed electrode can be manufactured by using a method of forming a mold of soluble polymers or the like at a part except for a part of a substrate surface at which a pattern is formed and filling the mold with metal and then dissolving only the mold. When a solution process is difficult to perform, laser etching or plasma etching can be used to form a certain pattern of metal remaining on the substrate.

According to the electrode for an enzymatic biofuel cell according to the embodiment of the present invention, the enzyme 140 may not be fixed to the substrate 110, but selectively fixed only to the electrode 120 by the metal-immobilized peptide 130.

Consequently, when the metal-immobilized peptide expressed at the enzyme is fixed on the pattern of the electrode for an enzymatic biofuel cell according to the embodiment of the present invention, an enzyme pattern can be formed along a shape of an electrode pattern.

The metal fulfills various functions in vivo, and about 30% of proteins are found in a state of having metal ions. In particular, Mg, Zn, Fe, Mn, or the like is often found in a state of binding to proteins. Fe present in hemoglobin or nitrogen-fixing enzymes is well known; besides, metal ions even function as a mediator in self-assembly of proteins.

In the present invention, the enzyme 140, in which the metal-immobilized peptide 130 is expressed, can be fixed to the electrode 120 by metal-peptide binding.

In this case, the enzyme 140 can contain an α sub-unit at which an active site is positioned and a γ sub-unit coupled to the α sub-unit, and the peptide can be expressed at either the a sub-unit or the γ sub-unit.

In this case, the peptide 130 is expressed at the α sub-unit or the γ sub-unit, and thereby the active site of the enzyme can be fixed to the electrode closely.

In this case, the peptide 130 can specifically bind to the electrode 120 and can have a length of about 0.1 nm.

In this case, the enzyme 140 can be fixed by binding of the peptide 130 in the enzyme which is performed through forming a microelectronic and micromagnetic film on a surface of the electrode 120.

In this case, the peptide 130 can contain 12 to 60 amino acids.

In this case, when the peptide 130 contains less than 12 amino acids, a problem can arise in that a distance required to transfer electrons between the electron transferring active site and the electrode is not secured due to a reduction in binding force between the peptide and the metal.

In this case, when the peptide 130 contains more than 60 amino acids, efficiency of electron transfer can be lowered due to an increase in distance between the electron transferring active site and the electrode by an increase in length and volume of the peptide.

In this case, when the peptide 130 contains more than 60 amino acids, the peptide can cover the electron transferring active site due to an increase in length and volume of the peptide, and thus an increase in electron transfer resistance can lead to a reduction in efficiency of an enzyme electrode.

Desirably, the peptide 130 can contain 12 to 24 amino acids.

In this case, when the peptide 130 contains less than 12 amino acids, a problem can arise in that a distance required to transfer electrons between the electron transferring active site and the electrode is not secured due to a reduction in binding force between the peptide and the metal.

In this case, when the peptide 130 contains more than 24 amino acids, efficiency of electron transfer can be lowered due to an increase in distance between the electron transferring active site and the electrode by an increase in length and volume of the peptide.

In this case, when the peptide 130 contains more than 24 amino acids, the peptide can cover the electron transferring active site due to an increase in length and volume of the peptide, and thus an increase in electron transfer resistance can lead to a reduction in efficiency of the enzyme electrode.

In this case, the metal-immobilized peptide 130 can contain one or more amino acid sequences of Sequence Number 1 to Sequence Number 10.

On the other hand, an electrode for an enzymatic biofuel cell in the related art has a first problem in that a binding position of an enzyme to a support body is non-specifically formed at an enzyme support body, and thus phases of a redox active site and an electron transferring active site of the enzyme are not adjusted, a second problem in that the electron transferring active site does not come into contact with the electrode by a distance of 2 nm or shorter, and a third problem in that the enzyme non-specifically binds to the electrode, and thus enzymes are not uniformly distributed on a surface of the electrode.

However, according to the embodiment of the present invention, the above-described problems can be solved because the metal-immobilized peptide can be selectively expressed at a specific position of the enzyme and have metal selectivity at a binding position depending on metal-peptide binding.

For example, the metal-immobilized peptide can contain one or more amino acid sequences of Sequence Number 1 to Sequence Number 10.

(Sequence Number 1)
LKAHLPPSRLPS (Gold)
(Sequence Number 2)
MHGKTQATSGTIQS (Gold)
(Sequence Number 3)
AYSSGAPPMPPF (Silver)
(Sequence Number 4)
IRPAIHIIPISH (Silver)
(Sequence Number 5)
MSPHPHPRHHHT (Silica)
(Sequence Number 6)
RKLPDAPGMHTW (Titanium)
(Sequence Number 7)
KLHSSPHTLPVQ (Cobalt)
(Sequence Number 8)
HSVRWLLPGAHP (Cobalt)
(Sequence Number 9)
CTLHVSSYC (Platinum)
(Sequence Number 10)
CPTSTGQAC (Platinum)

In this case, the enzyme can include a glucose dehydrogenase, a glucose oxidase, an alkaline phosphatase, or a carbon monoxide dehydrogenase.

In this case, the enzyme can further include a cofactor.

In this case, the cofactor can be added to improve catalytic activity of the enzyme.

For example, the cofactor can contain flavin adenine dinucleotide (FAD) or nicotinamide adenine dinucleotide (NAD). However, the cofactor is not limited thereto, and the enzyme can contain an appropriate cofactor, as necessary.

### Manufacturing Example 1

In order to manufacture the electrode for an enzymatic biofuel cell according to the embodiment of the present invention, an FAD-GDH (glucose dehydrogenase) derived from Burkholderia cepacia (Genbank ID: AF430844.1) in which a metal-immobilized peptide was expressed, was manufactured.

The FAD-GDH was manufactured through a general polymerase chain reaction (PCR) method using pET21a plasmid.

In this case, the pET21a plasmid had six histidine tags, and a maltose binding protein (MBP) was inserted in a C-terminus of each of the histidine tags for separation of the enzyme after manufacturing of the enzyme. In addition, a tobacco etch virus (TEV) gene was inserted between a GDH gene and the MBP for separation of the enzyme after performing PCR.

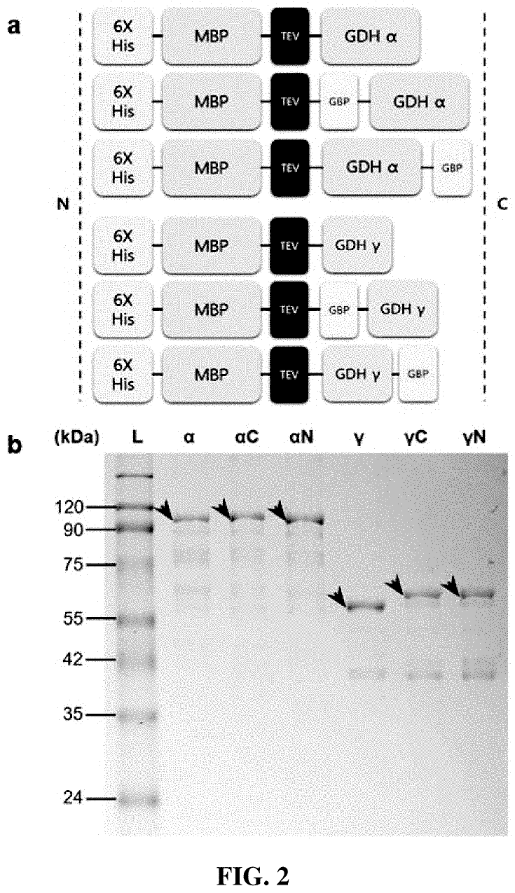

In this case, a gold binding peptide (GBP) as a metal-immobilized peptide was inserted in an N-terminus of an α unit, a C-terminus of the α unit thereof, an N-terminus of a γ unit thereof, or a C-terminus of the γ unit of the GDH, and this is illustrated in FIG. 2.

In this case, an amino acid sequence of the GBP was LKAHLPPSRLPS and had 1.78 kDa and a length of 0.1 nm.

Here, a in FIG. 2 is a schematic diagram illustrating plasmid used in Manufacturing Example 1.

In addition, b in FIG. 2 is a 12% SDS-gel photograph of an electrophoresed enzyme manufactured in Manufacturing Example 1.

With reference to FIG. 2, it is possible to find that, when compared to a column of an α unit and a column of a γ unit at which the metal-immobilized peptide is not expressed, bands have a larger mass at an αC column indicating a case where a metal-immobilized peptide is expressed at the C-terminus of the α unit, an αN column indicating a case where a metal-immobilized peptide is expressed at the N-terminus of the α unit, a γC column indicating a case where a metal-immobilized peptide is expressed at the C-terminus of the γ unit, and a γN column indicating a case where a metal-immobilized peptide is expressed at the N-terminus of the γ unit. In addition, it is possible to confirm that the αC column and the αN column have the same band position as each other and the γC column and the γN column have the same band position as each other, and this means that the enzymes according to Manufacturing Example 1 are properly manufactured.

### Manufacturing Example 2

First, a silicon wafer substrate was coated with gold (Au) by a size of 1 cm².

Next, a 0.5 uM solution of FAD-GDH, at which the GBP was expressed, was manufactured.

Next, an electrode for an enzymatic biofuel cell was manufactured by immersing the substrate coated with Au in the solution for 20 min. and immobilizing the GBP at an Au surface.

### Comparative Example 1

An electrode for an enzymatic biofuel cell was manufactured in the same way as in Manufacturing Example 2, except that the GBP was not expressed.

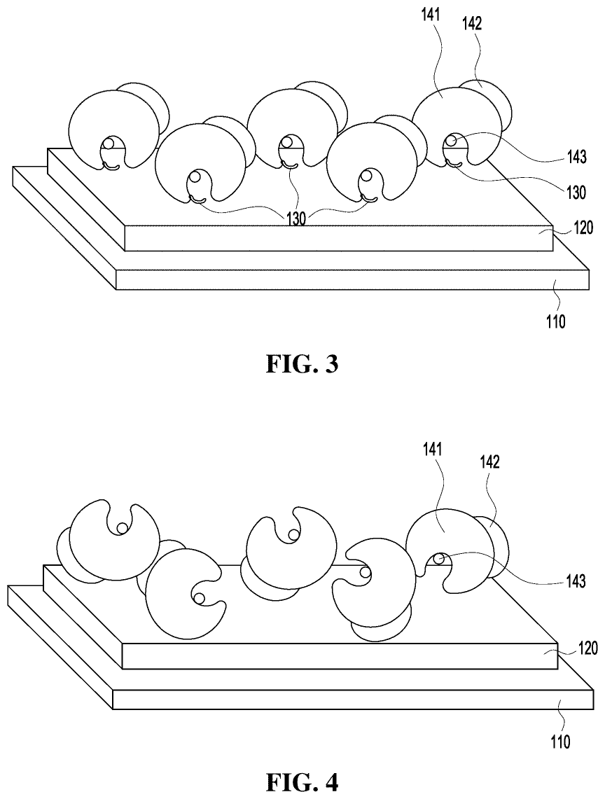

FIG. 3 is a schematic view illustrating the electrode for an enzymatic biofuel cell according to the embodiment of the present invention.

With reference to FIG. 3, the enzymatic biofuel cell according to the embodiment of the present invention can include the substrate 110, the electrode 120 positioned on the substrate 110, the metal-immobilized peptide 130 immobilized at the electrode 120, an α unit 141 of a glucose dehydrogenase at which the peptide 130 is expressed, a γ unit 142 of a glucose dehydrogenase which is coupled to the α unit 141 of the glucose dehydrogenase, and a cofactor 143 of a glucose dehydrogenase which is coupled to the α unit 141 of the glucose dehydrogenase.

FIG. 4 is a schematic view illustrating an electrode for an enzymatic biofuel cell in the related art.

With reference to FIG. 4, the biofuel cell in the related art can include a substrate 110, an electrode 120 positioned on the substrate 110, an α unit 141 of a glucose dehydrogenase positioned on the electrode 120, a γ unit 142 of a glucose dehydrogenase which is coupled to the α unit 141 of the glucose dehydrogenase, and a cofactor 143 of a glucose dehydrogenase which is coupled to the α unit 141 of the glucose dehydrogenase.

With reference to FIGS. 3 and 4, it is possible to find that the electrode for an enzymatic biofuel cell according to the embodiment of the present invention has a configuration in which the metal-immobilized peptide 130 causes an active site present at the α unit 141 of the glucose dehydrogenase to be fixed to the electrode 120 closely. On the other hand, it is possible to find that the electrode for an enzymatic biofuel cell in the related art has a configuration in which the α unit 141 of the glucose dehydrogenase is not fixed by the metal-immobilized peptide 130, and thus a distance between an active site present at the α unit 141 and the electrode 120 is not constant and is longer than that in a case of the electrode for an enzymatic biofuel cell according to the embodiment of the present invention.

The enzymatic biofuel cell generates an electromotive force by using hydrogen ions or electrons generated by a chemical reaction occurring at the active site of the enzyme. In this case, in order to improve performance of the enzymatic biofuel cell, it is important to effectively transfer the electrons generated at the active site of the enzyme to the electrode. Here, in order to effectively transfer the electrons generated at the active site of the enzyme to the electrode, it is important to shorten a distance between the active site of the enzyme and the electrode.

The electrode for an enzymatic biofuel cell according to the embodiment of the present invention has a configuration in which the metal-immobilized peptide 130 is expressed at the a unit 141 at which the active site of the enzyme is positioned or at the γ unit 142 close to the α unit 141 and is directly immobilized at the electrode, and thereby the active site and the electrode are fixed to each other by a close distance.

Consequently, the electrode for an enzymatic biofuel cell according to the embodiment of the present invention has a configuration in which the enzyme is directly fixed to the electrode by the peptide expressed at the enzyme, and thereby the distance between the active site of the enzyme and the electrode is shortened such that the performance of the enzymatic biofuel cell can improve.

In addition, the electrode for an enzymatic biofuel cell according to the embodiment of the present invention can provide a pattern-formed electrode. This is because the metal-immobilized peptide specifically binds to a metal. Consequently, the metal-immobilized peptide is fixed on the pattern-formed electrode. In this manner, a pattern-formed electrode for an enzymatic biofuel cell can be formed. Consequently, the electrode can be formed by a method in which a metal is formed by nanoparticles and is fixed on the substrate.

The glucose dehydrogenase contains an α unit which binds to the flavin adenine dinucleotide (FAD), a β unit which is cytochrome c, and a γ unit which is a chaperone analog. The FAD fulfills a function of mediating electron transfer by binding to a glucose oxidase or a glucose dehydrogenase mainly in vivo. The FAD is considered as the same body as the enzyme since the FAD is essential for reactions of enzymes, and a binding form of the FAD to the glucose dehydrogenase is referred to as FAD-GDH and is considered as one enzyme. The cytochrome c is a protein present in a large number of species and can be found in a plant, an animal, and many unicellular animals. The cytochrome c is a protein having ham molecules, is formed by about 100 amino acids, and has a molecular weight of 12,000. The cytochrome c fulfills a function of transferring electrons between a complex III and a complex IV of an in-vivo electron transfer system and adjusting apoptosis. The chaperone is involved in folding and transforming of the protein and influences binding of the enzyme and the substrate and emission of a product.

The glucose dehydrogenase is synthesized to express the peptide. The peptide is expressed at either the α unit or the γ unit. The peptide is inserted into the N-terminus or the C-terminus of the α unit or the γ unit of a gene of the dehydrogenase. In this case, when the peptide is immobilized at the metal depending on selection of the α unit or the γ unit or the N-terminus or the C-terminus of the units, a distance between the glucose dehydrogenase and the metal can be controlled.

When the peptide is expressed at the glucose dehydrogenase and the peptide is immobilized at the electrode, the glucose dehydrogenase can directly transfer electrons to the electrodes.

A method in which an enzyme transfers an electron to an electrode can be divided into a mediated electron transfer (MET) method and a direct electron transfer (DET) method; however, a problem arises in the MET method in that an electron potential is lowered due to an intermediate mediator. This is due to the fact that while electron transfer distance is important for efficient electron transfer, the electron transfer distance increases due to the intermediate mediator in the MET method. In the present invention, since the peptide expressed at the glucose dehydrogenase is directly fixed at the electrode, the glucose dehydrogenase can be very closely fixed to the electrode. Hence, the DET method can be conducted, and thus a high electron potential can be maintained. The electron transfer efficiency depending on the electron transfer distance can be determined by the following expression (1).

$${K}_{et} = {10}^{13} {e}^{- 0.91 ( d - 3 )} {e}^{[ \frac{- ( Δ G + λ )}{4 RT λ} ]} \qquad Expression ( 1 )$$

(In Expression (1), Kₑₜ represents an electron transfer rate constant, d represents an actual electron transfer distance, G represents free energy, and λ represents reconstruction energy.)

Consequently, the electrode for an enzymatic biofuel cell according to the embodiment of the present invention has a configuration in which the active site of the enzyme and the electrode are fixed to be close to each other by the metal-immobilized peptide, and thereby the electron transfer efficiency can improve.

### Experimental Example 1

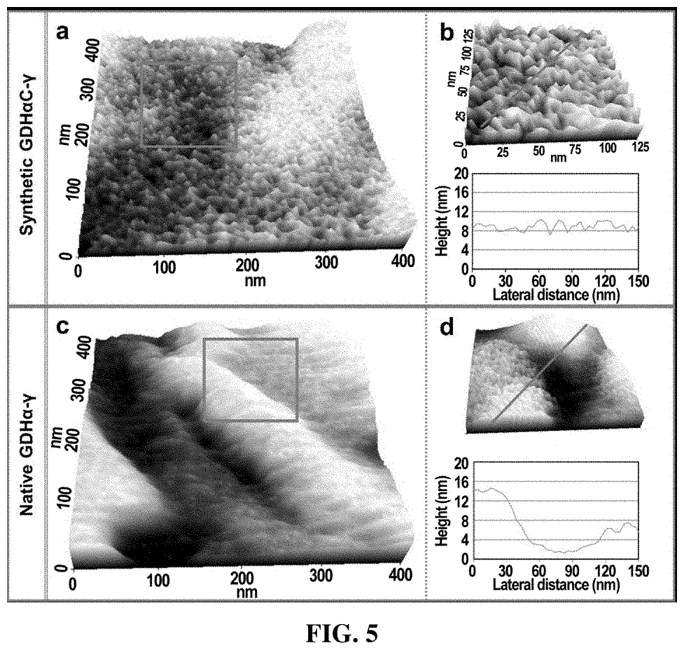

FIG. 5 illustrates results from AFM analysis of surfaces of electrodes manufactured in Manufacturing Example 2 and Comparative Example 1 under conditions of a 125 um silicon/aluminum cantilever, a resonance frequency of 200 to 400 kHz, a nominal force constant of 42 N/m, and a maximum scan speed of 0.2 Hz.

FIG. 5 illustrates AFM measurement graphs of the manufacturing example and the comparative example according to the present invention.

In FIG. 5, a is an AFM measurement graph of the electrode for an enzymatic biofuel cell manufactured by Manufacturing Example 2, b illustrates an AFM graph of an inner part of a red box in a of FIG. 5 and a graph illustrating a line-based electrode height deviation represented by a red line, c is an AFM measurement graph of the electrode for an enzymatic biofuel cell manufactured by Comparative Example 1, and d illustrates an AFM graph of an inner part of a red box in c of FIG. 5 and a graph illustrating a line-based electrode height deviation represented by a red line.

With reference to FIG. 5, it is possible to find that the surface height deviation in Manufacturing Example 2 is smaller than that in Comparative Example 1. It is determined that Manufacturing Example 2 has the smaller height deviation because the GBP, which is the metal-immobilized peptide, is expressed, and thus the FAD-GDHs are more uniformly fixed to the surface of the electrode without conglomerate, whereas Comparative Example 1 has bigger height deviation as a lateral distance increases because the GBP is not expressed, and thus the FAD GDHs are not uniformly distributed over the surface of the electrode.

### Experimental Example 2

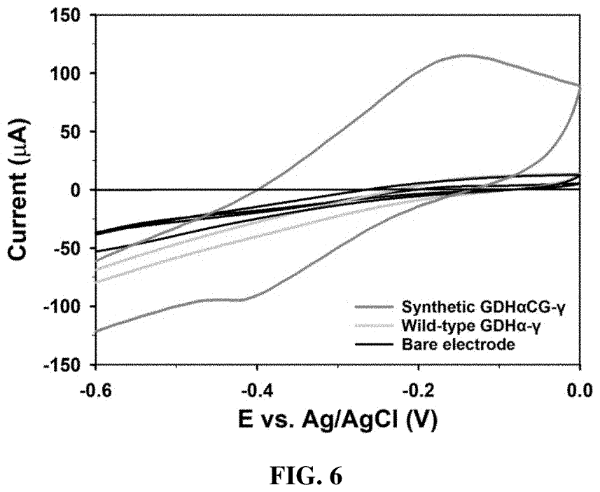

FIG. 6 illustrates a cyclic voltammogram measured in a buffer containing 100 mM glucose by configuring a three-electrode system in which the electrodes manufactured in Manufacturing Example 2 and Comparative Example 1 are each used as active electrodes, a Pt wire is used as a counter electrode, Ag/AgCl is used as a reference electrode.

FIG. 6 illustrates a cyclic voltammogram of the manufacturing example and the comparative example according to the present invention.

With reference to FIG. 6, it is possible to find that a current value in Manufacturing Example 2 is much larger than that in Comparative Example 1. This is because a distance between the active site of the FAD-GDH and the surface of the electrode is shortened due to expression of the GBP such that the FAD-GDH can directly transfer the electron, and thus the electrode efficiency improves.

### Experimental Example 3

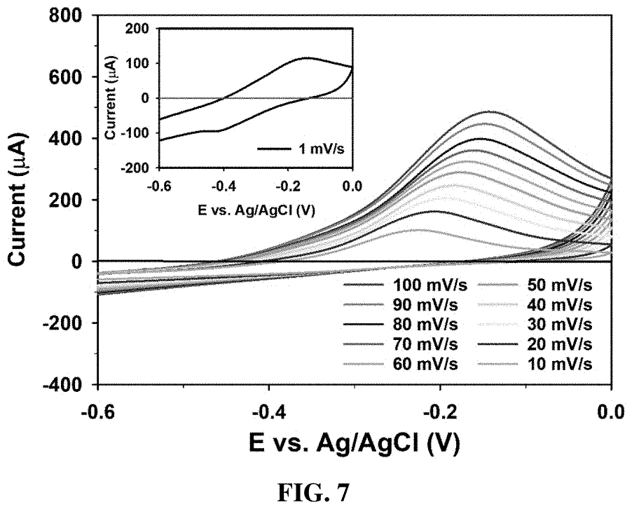

FIG. 7 illustrates a cyclic voltammogram measured in a buffer containing 100 mM glucose by varying scan speed at 1 mV/s, 20 mV/s, 40 mV/s, 60 mV/s, 80 mV/s 100 mV/s, after configuring a three-electrode system in which the electrode manufactured in Manufacturing Example 2 is used as an active electrode, a Pt wire is used as a counter electrode, Ag/AgCl is used as a reference electrode.

FIG. 7 illustrates a cyclic voltammogram based on a scan speed according to the present invention.

With reference to FIG. 7, it is possible to find that a current value in Manufacturing Example 2 increases as the scan speed increases. This means that as the scan speed increases, direct electron transferring from the FAD-GDH to the electrode increases.

The electrode for an enzymatic biofuel cell according to the embodiment of the present invention can have a decrease in distance between the active site of the enzyme and the electrode, and thereby electrons generated at the active site are effectively transferred to the electrode such that the performance of the enzymatic biofuel cell can improve.

The electrode for an enzymatic biofuel cell according to the embodiment of the present invention can have a configuration in which the metal-immobilized peptide which is specifically fixed at the metal can be used as an enzyme fixing means, and thereby the enzyme can be selectively fixed only to a metal electrode.

The electrode for an enzymatic biofuel cell according to the embodiment of the present invention can have a configuration in which the metal-immobilized peptide which is specifically fixed at the metal can used as an enzyme fixing means, and thereby the enzyme electrode can be patterned.

The electrode for an enzymatic biofuel cell according to the embodiment of the present invention can have a configuration in which the metal-immobilized peptide which is expressed at the enzyme can be used as an enzyme fixing means, and thereby the enzymes can be uniformly fixed without conglomerate at one part of the electrode.

Hereinafter, an enzymatic biofuel cell according to another embodiment of the present invention will be described.

In this case, the enzymatic biofuel cell includes the electrode for an enzymatic biofuel cell according to the embodiment of the present invention.

In this case, the enzymatic biofuel cell can include an anode electrode, an electrolyte layer positioned on the anode electrode, and a cathode electrode positioned on the electrolyte layer.

In this case, the electrode for an enzymatic biofuel cell can be used as the anode electrode or the cathode electrode.

The enzymatic biofuel cell according to the embodiment of the present invention can have a decrease in distance between the active site of the enzyme and the electrode, and thereby electrons generated at the active site are effectively transferred to the electrode such that the performance of the enzymatic biofuel cell can improve.

The enzymatic biofuel cell according to the embodiment of the present invention can have a configuration in which the metal-immobilized peptide which is specifically fixed at the metal can be used as an enzyme fixing means, and thereby the enzyme can be selectively fixed only to a metal electrode such that a patterned electrode can be formed.

The enzymatic biofuel cell according to the embodiment of the present invention can have a configuration in which the metal-immobilized peptide which is expressed at the enzyme can be used as an enzyme fixing means, and thereby the enzymes can be uniformly fixed without conglomerate at one part of the electrode such that the efficiency of the cell can improve.

The description of the present invention described above is provided as an example, and a person of ordinary skill in the art to which the present invention belongs can understand that it is possible to easily modify the present invention to another embodiment without changing the technical idea or an essential feature of the present invention. Therefore, the embodiments described above need to be understood as exemplified embodiments in every aspect and not as embodiments to limit the present invention. For example, configurational elements described in a single form can be realized in a distributed manner. Similarly, the configurational elements described in the distributed manner can be realized in a combined manner.

The scope of the present invention needs to be represented by the claims to be described below, and meaning and the scope of the claims and every modification or modified embodiment derived from an equivalent concept of the claims need to be construed to be included in the scope of the present invention.

### REFERENCE SIGNS LIST

110 SUBSTRATE
120 ELECTRODE
130 METAL-IMMOBILIZED PEPTIDE
140 ENZYME
141 α UNIT OF ENZYME
142 γ UNIT OF ENZYME
143 COFACTOR OF ENZYME
150 ENZYME PATTERN

1. An electrode for an enzymatic biofuel cell, comprising:
a substrate;
an electrode positioned on the substrate; and
an enzyme pattern containing an enzyme in which a metal-immobilized peptide is expressed positioned on the electrode,
wherein the metal-immobilized peptide expressed from the enzyme is fixed at the electrode.

2. The electrode for an enzymatic biofuel cell according to claim 1,
wherein a distance between an electron transferring active site of the enzyme and a surface of the electrode is 2 nm or shorter.

3. The electrode for an enzymatic biofuel cell according to claim 1,
wherein the substrate contains a silicon wafer, conductive polymers, carbon cloth, carbon paper, or graphene.

4. The electrode for an enzymatic biofuel cell according to claim 1,
wherein the electrode contains silica, Cu, Zn, Fe, Ni, Co, Mn, Au, or Ag.

5. The electrode for an enzymatic biofuel cell according to claim 1,
wherein the enzyme contains an α unit at which an active site is positioned and a γ unit coupled to the α unit, and
wherein the peptide is expressed at either the α unit or the γ unit.

6. The electrode for an enzymatic biofuel cell according to claim 1,
wherein the peptide specifically binds to the electrode and has a spiral structure.

7. The electrode for an enzymatic biofuel cell according to claim 1,
wherein the peptide contains 12 to 60 amino acids.

8. The electrode for an enzymatic biofuel cell according to claim 1,
wherein the peptide contains one or more amino acid sequences of Sequence Number 1 to Sequence Number 10.

9. The electrode for an enzymatic biofuel cell according to claim 1,
wherein the enzyme includes a glucose dehydrogenase, a glucose oxidase, an alkaline phosphatase, or a carbon monoxide dehydrogenase.

10. The electrode for an enzymatic biofuel cell according to claim 1,
wherein the enzyme further includes a cofactor.

11. An enzymatic biofuel cell comprising the electrode according to claim 1.
